(19) United States
(12) Patent Application Publication
(10) Pub. No.: US 2023/0001137 A1
(43) Pub. Date: Jan. 5, 2023

(54) LIGHT-DIFFUSING ELEMENT CONFIGURED TO AFFECT THROMBI FORMATION ON INTRAVENOUS CATHETER

(71) Applicant: CORNING INCORPORATED, CORNING, NJ (US)

(72) Inventor: William Spencer Klubben, III, Boston, MA (US)

(21) Appl. No.: 17/783,362

(22) PCT Filed: Dec. 3, 2020
(86) PCT No.: PCT/US2020/062963

§ 371 Date: Jun. 8, 2022

Related U.S. Application Data
(60) Provisional application No. 62/947,099, filed on Dec. 12, 2019.

(51) Int. Cl. A61M 25/00; A61N 5/06

(52) U.S. Cl. CPC A61M 25/0017; A61M 25/0045; A61N 5/062; A61M 2025/0019; A61N 2005/0602; A61N 2005/063; A61N 2005/0659; A61N 2005/0652; A61N 2005/0661; A61N 2005/0662

(57) ABSTRACT

Disclosed are embodiments of a method for affecting thrombi formation on an indwelling catheter. The method involves the step of providing an intravenous catheter. The intravenous catheter includes an inner surface and an outer surface, and the intravenous catheter is located within a blood vessel. A light diffusing element is inserted into the intravenous catheter. Light is emitted from the light diffusing element such that the light irradiates the intravenous catheter. The light emitted from the light diffusing element is configured to promote or hinder thrombi formation on the inner surface or outer surface of the intravenous catheter. Also disclosed are an illumination system for affecting thrombi formation on an intravenous catheter as well as an indwelling intravenous catheter system.

### CROSS-REFERENCE TO RELATED APPLICATIONS

This application claims the benefit of priority under 35 U.S.C § 120 of U.S. Provisional Application Ser. No. 62/947,099 filed on Dec. 12, 2019 the content of which is relied upon and incorporated herein by reference in its entirety.

### BACKGROUND

The present disclosure relates to method and system for preventing or treating thrombus and, in particular, to a method and system of preventing thrombus from developing in or embolus releasing from an intravenous catheter. Many medical devices come in contact with whole blood or are inserted into the vascular system. When foreign objects are inserted into a vein or artery a thrombus (i.e., a blood clot) can form. Thrombus formation is problematic because sometimes it can create an embolus (a blot clot traveling through the blood stream) which can move to the brain and cause a stroke, to the heart and cause a myocardial infarction (heart attack), or to other organs to cause organ dysfunction. In certain circumstances, chemical agents are injected into the bloodstream, e.g. blood thinners, to prevent coagulation; however, these pharmaceuticals have other potentially undesirable consequences and/or side effects as well.

### BRIEF SUMMARY

According to an aspect, embodiments of the present disclosure relate to a method for affecting thrombi formation on an indwelling catheter. The method involves the step of providing an intravenous catheter. The intravenous catheter includes an inner surface and an outer surface, and the intravenous catheter is located within a blood vessel. A light diffusing element is inserted into the intravenous catheter. Light is emitted from the light diffusing element such that the light irradiates the intravenous catheter. The light emitted from the light diffusing element is configured to promote or hinder thrombi formation on the inner surface or outer surface of the intravenous catheter.

According to another aspect, embodiments of the present disclosure relate to an illumination system. The illumination system includes a light source configured to emit light having a wavelength of from 200 nm to 2000 nm. The illumination system also includes a light diffusing element optically coupled to the light source. The light diffusing element is configured to receive the light emitted by the light source and diffuse the light along a length thereof. The light diffusing element is configured to be inserted into, embedded in, or attached to an intravenous catheter located in a blood vessel. The light diffused from the light diffusing element is configured to promote or hinder the formation of thrombi on an inner surface or an outer surface of the intravenous catheter.

According to still another aspect, embodiments of the present disclosure relate to an indwelling catheter system. The catheter system includes a catheter configured for insertion into a blood vessel. The catheter has an inner surface and an outer surface. The inner surface defines a central bore extending along a longitudinal axis of the catheter. A light diffusing element is disposed within the central bore of the catheter. A light source is optically coupled to the light diffusing element and configured to emit light. The light diffusing element is configured to receive the light emitted by the light source and diffuse the light along a length thereof. Further, the light diffused from the light diffusing element is configured to promote or hinder thrombi formation on the inner surface or the outer surface of the catheter.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiment(s), and together with the description serve to explain principles and operation of the various embodiments.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 is a schematic illustration of an illumination system for preventing thrombi or emboli in an intravenous catheter within a blood vessel, according to an exemplary embodiment;

FIG. 2 is a schematic illustration of a cross-section of a blood vessel containing a catheter and a light diffusing element, according to an exemplary embodiment;

FIG. 3 is a flow diagram of a method of preventing thrombi or emboli in an intravenous catheter, according to an exemplary embodiment; and

FIG. 4 is a schematic illustration of a longitudinal cross-section of a light-diffusing optical fiber, according to an exemplary embodiment.

### DETAILED DESCRIPTION

Various embodiments of systems and methods for preventing the formation of thrombi on an intravenous catheter or preventing thrombi from breaking away from an intravenous catheter are provided herein. As will be described below, the illumination system includes a light diffusing element inserted into the intravenous catheter. The light diffusing element is optically coupled to a therapeutic light source that emits light having a wavelength that hinders or promotes the formation of thrombi. By hindering the formation of thrombi, such thrombi are prevented from developing in the first place, which avoids the possibility of emboli formation. By promoting the formation of thrombi, the size of the thrombi can be reduced by completing the clotting reaction quicker, and further, the thrombi can be made stronger, which helps ensure that the thrombi do not break away from the intravenous catheter. Advantageously, the light diffusing element, such as a light diffusing fiber, can be reversibly inserted into the intravenous catheter without modification of the catheter, and the light diffusing element can provide targeted treatment of thrombi. Conventionally, thrombi were treated using pharmaceuticals, such as blood thinner medications, which may have undesired consequences (such as difficulty with wound healing in other parts of the body). Applicant believes that treatment of thrombi with the light diffusing element will avoid such undesired consequences. These and other advantages will be described more fully below in relation to the exemplary embodiments discussed herein and shown in the figures. These embodiments are presented by way of illustration and not by way of limitation.

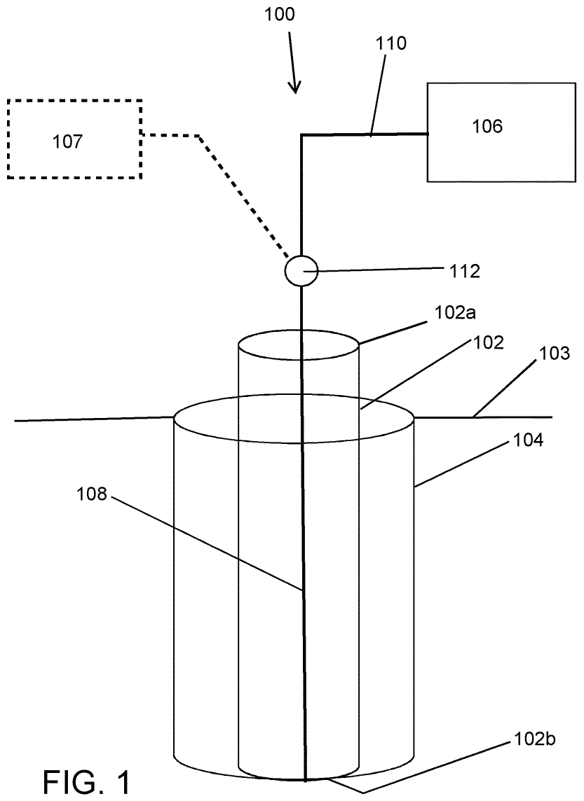

FIG. 1 is a schematic depiction of an illumination system 100 for an intravenous catheter 102. The intravenous catheter 102 is shown schematically within a patient's body 103 in a blood vessel 104. In an embodiment, the intravenous catheter 102 is an in-dwelling catheter, meaning that the catheter is configured to spend an extended period of time in the blood vessel 104 (i.e., it is not removed between uses). In this way, the catheter 102 can be used to deliver treatments to a patient 103 via the patient's blood stream, or the catheter 102 can be used to periodically withdraw samples from the patient's body 103 via the patient's blood stream. In this regard, the intravenous catheter 102 has a first end 102a that is exterior to a patient's body 103 a second end 102b that is interior to the blood vessel 104 within a patient's body 103. The illumination system 100 is configured such that the possibility of thrombi forming on or in the intravenous catheter 102 or an emboli breaking free from the intravenous catheter 102 is reduced.

The illumination system 100 includes a therapeutic light source 106 optically coupled to a light diffusing element 108. In embodiments, the light diffusing element 108 is a glass or plastic light-diffusing optical fiber (LDF). In other embodiments, the light diffusing element 108 may consist of periodically spaced light emitting diodes (LED) along a wire. In embodiments, the therapeutic light source 106 may comprise any light source structurally configured to emit light, for example, a laser light source, a light emitting diode (LED), a laser diode, an incandescent lamp, an ultraviolet light source, such as an ultraviolet LED, an ultraviolet lamp, or the like. In an embodiment, the light diffusing element 108 is optically coupled directly to the light source 106. In other embodiments, the light diffusing element 108 is optically coupled to a transmission fiber 110, which is coupled to light source 106. In embodiments, the transmission fiber 110 is optically coupled to the light diffusing element 108 using an optical coupling 112 (e.g., a mechanical or fusion splice). Further, in embodiments, the illumination system may comprise additional therapeutic light sources, for example, a second therapeutic light source 107 such that the light diffusing element 108 may be selectively optically coupled to different light sources 106, 107 outputting different wavelengths of light.

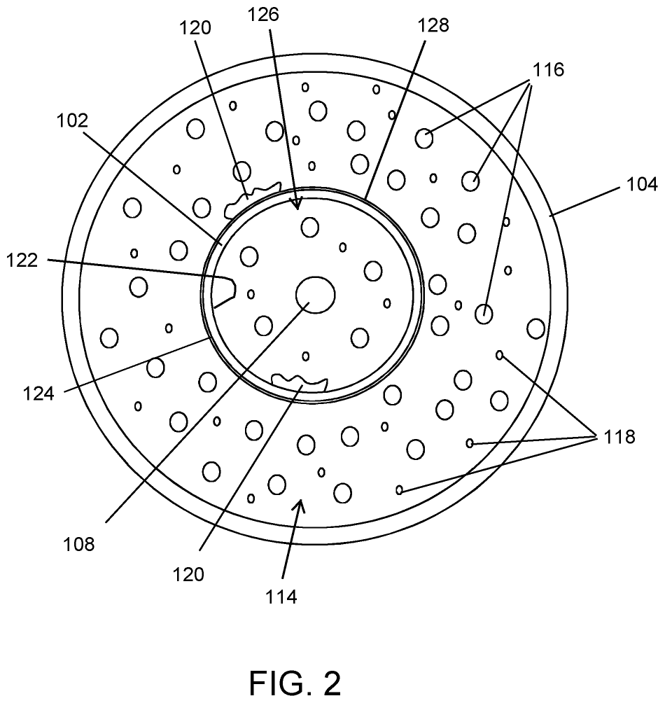

FIG. 2 depicts a cross-section of the blood vessel 104 containing the light diffusing element 108 within the intravenous catheter 102. The blood vessel 104 has blood 114 running therethrough. For the purposes of this discussion, the blood 114 is comprised of red blood cells 116 and platelets 118 (in reality, blood 114 contains other components, such as white blood cells and plasma, which are not specifically depicted). Platelets 118 initiate the clotting action of blood 114 by aggregating at a wound site. In particular, the aggregated platelets 118 form a mesh of cross-linked fibrin protein with the red blood cells 116. Generally, this clotting action takes place to stem the flow of blood from a wound in tissue. However, the platelets 118 may also bind to an indwelling catheter, such as the intravenous catheter 102, and form a thrombus 120.

The intravenous catheter 102 has an interior surface 122 and an exterior surface 124. The interior surface 122 defines an internal bore 126. In the embodiment of FIG. 2, the light diffusing element 108 is shown disposed within the internal bore 126. However, in other embodiments, the light diffusing element 108 may be embedded in the catheter 102 in the material between the interior surface 122 and the exterior surface 124. Further, in embodiments, the light diffusing element 108 or elements 108 may be attached to the interior surface 122 and/or exterior surface 124 of the catheter 102. Thrombi 120 can develop on the interior surface 122 or exterior surface 124 of the intravenous catheter 102. The formation of thrombi 120, especially small thrombi 120, is not necessarily, in and of itself, the ultimate concern. Instead, a potentially dangerous condition occurs if a thrombus 120 forms to a large size and breaks away from the intravenous catheter 102, which is referred to as embolus. The embolus can block blood vessels 104, leading to stroke, heart attack, or other organ dysfunctions depending on where the embolus lodges.

According to the present disclosure, the light diffusing element 108 attached to, embedded in, or inserted into the catheter 102 emits light of a wavelength that affects (i.e., promotes or hinders) the formation of thrombi 120. For example, the light may hinder the formation of thrombi 120 so that they do not develop at all, or the light may promote the formation of thrombi 120 so that the thrombi 120 harden quickly and not break away from the intravenous catheter 102. In particular, the light diffusing element 108 irradiates the interior surface 122 of the intravenous catheter 102 to promote or hinder the formation of thrombi 120 on the interior surface 122 of the intravenous catheter 102. Further, in embodiments, the intravenous catheter 102 is made from a material that is transparent to the light diffused from the light diffusing element 108. Thus, the light diffusing element 108 may irradiate the exterior surface 124 with light transmitted through the intravenous catheter 102 from the interior surface 122 to the exterior surface 124, or vice versa.

As will be discussed below, the emitted light can affect thrombi formation 120 in a variety of different ways. In operation, the therapeutic light source 106 may emit light that is diffused by the light diffusing element 108. In embodiments, the emitted light has a wavelength between about 200 nm and about 2000 nm, for example between about 200 nm and 400 nm, between about 800 nm and 2000 nm, between about 400 nm and 800 nm, or between about 400 nm and 1310 nm. In embodiments, the particular wavelength of light may be selected to directly affect the reactions leading to the formation of thrombi 120. In other embodiments, the therapeutic light source 106 may emit light configured to be diffused from the light diffusing element 108 at wavelengths that activates, alters, or otherwise reacts with one or more photo-active pharmaceuticals, i.e., photosensitizers, or that activates, alters, or otherwise reacts with a photo-active coating or coatings.

Further, the therapeutic light source 106 may emit a pulsed light. In embodiments, the pulses correspond to a differences between peak intensity and average intensity of the light emitted from the light diffusing element 108. For example, the pulsed light may be pulsed at frequency within a frequency range comprising between about 70 Hz and 80 Hz, between about 145 Hz and 155 Hz, between about 290 Hz and 300 Hz, between about 585 Hz and 595 Hz, between about 1170 Hz and about 1180 Hz, between about 2345 Hz and about 2355 Hz, or between about 4695 Hz and about 4705 Hz. Further, the treatment frequency range may encompass one or more of the Noiger frequencies, for example, 73 Hz, 147 Hz, 294 Hz, 587 Hz, 1174 Hz, 2349 Hz, 4698 Hz, or the like.

In a particular embodiment, the light diffusing element 108 emits ultraviolet (UV) light, such as light having a wavelength of about 200 nm to about 400 nm. Because UV light is a high energy form of light, it has the ability to form and break bonds. In embodiments, UV light is used to locally prevent or pre-break bonds between platelets 118 to prevent formation of large thrombi 120. In other embodiments, UV light is used to locally promote and propagate biomolecule polymerization to create a stronger thrombi 120 that would be less likely to break down over time, thus reducing the risk of an embolism (the occlusion of a blood vessel 104 by an embolus).

In another particular embodiment, the light diffusing element 108 is configured to emit infrared (IR) light, such as light having a wavelength of about 800 nm to about 2000 nm, which has been shown to have wound healing capabilities. Thus, in embodiments, the wound healing capabilities decrease the thrombus 120 formation by speeding up the healing process, which reduces the size of the thrombus 120 developed. Additionally, in embodiments, the IR light is used to strengthen the thrombus 120 bond formation and prevent emboli migration and embolism formation.

In still another particular embodiment, the light diffusing element 108 is configured for photodynamic therapy (PDT). In PDT, a light-activated biomolecule, or photosensitizer, is injected, ingested, or applied to a region of interest and illuminated with a certain wavelength of light. For example, Photofrin™ is a photosensitizer that may be activated by emitted light having wavelengths between about 600 nm and about 680 nm, or wavelengths near UV wavelengths, such as between about 370 nm and about 420 nm. Other photosensitizers usable with the light diffusing element 108 include 5-aminolaevulinic acid, verteporfin, tin ethyl etiopurpurin (Purlytin®), temoporfin (Foscan®), lutetium texaphyrin (Lutex®), ATMPn (9-acetoxy-2,7,12,17-tetrakis-(β-methoxyethyl)-porphycene), zinc phthalocyanine, and napthalocyanines, among others. The photosensitizer photo-reacts to the light from the light diffusing element 108 and releases a highly reactive form of oxygen which kills/damages the nearest cell, tissue, or structure. In embodiments, PDT is used to prevent the formation of thrombi 120 by breaking early-formed bonds and potentially destroying molecules or cells which create platelets 118. In embodiments, PDT is performed with light having a wavelength of from about 350 nm to about 800 nm.

In yet another particular embodiment, the light diffusing element 108 is configured for photobiomodulation (PBM), also known as low level laser therapy (LLLT). While the specific mechanism of PBM is not fully understood, low levels of laser light (e.g., having an intensity of 5 to 500 mW/cm²) have been demonstrated to have positive effects on a wide variety of biological functions, including wound healing, joint health, lung health, and brain health. By delivering the low levels of laser light locally, thrombi 120 and emboli can be prevented from forming. In embodiments, PBM is performed using light having a wavelength of from about 400 nm to about 1310 nm.

In still yet another particular embodiment, the light diffusing element 108 can be configured to activate a coating 128 containing a photo-active substance on the interior surface 122, the exterior surface 124, or both, or the intravenous catheter 102 can be impregnated with a photo-activated substance. When illuminated, the photo-activated substance can either prevent thrombi 120 formation or strengthen the bonds of the thrombi 120 to keep them from breaking away. In an embodiment, the photo-activates substance is a luminescent material. In embodiments, the luminescent material is fluorescent or phosphorescent. For example, upon being contacted by light having a first wavelength from the light diffusing element 108, the luminescent photo-active substance can emit light having a second wavelength. More particularly, in embodiments, the second wavelength can be UV or IR to provide the effects described above, and in other embodiments, the second wavelength can be configured for PDT or PBM. In another example, the photo-active substance can make the coating hydrophobic or hydrophilic. In embodiments, the coating is an oxide, e.g., an inorganic oxide, such as ZnO, TiO₂, or SnO₂. In other embodiments, the coating is a polymer that includes such functional groups as azobenzene, spiropyran, salicylideneaniline, or derivatives thereof. In general, the surface wettability of the coating is transitioned from hydrophilic to hydrophobic, or vice versa, by exposing the coating to UV light (e.g., in the range of 200 nm to 400 nm) or visible light (e.g., in the range of 400 nm to 800 nm). In this way, the thrombi 120 are prevented from attaching to the surface of the intravenous catheter 102 or are strongly held to the surface of the catheter so that emboli do not break free.

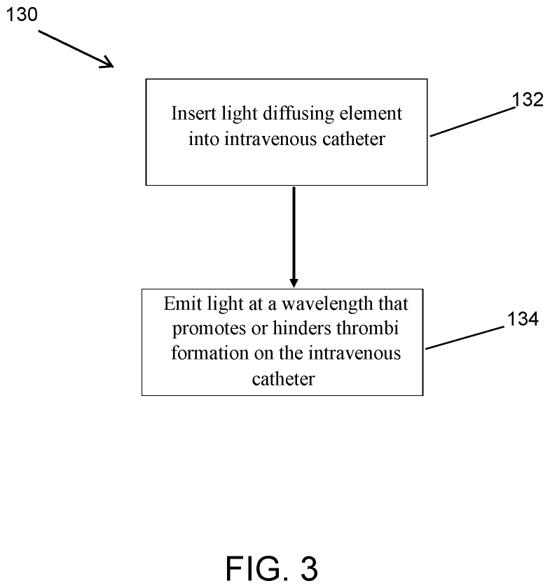

FIG. 3 depicts a flow diagram of a method 130 of preventing or strengthening thrombi 120. In a first step 132, the light diffusing element 108 is inserted into the intravenous catheter 102. In embodiments, the light diffusing element 108 is permanently fixed within the intravenous catheter 102 after insertion. In other embodiments, the light diffusing element 108 can be configured for reversible insertion into the intravenous catheter 102. That is, in the latter embodiment, the light diffusing element 108 can periodically be inserted into the intravenous catheter 102 to break-up or strengthen thrombi 120 and then taken back out of the intravenous catheter 102. In a second step 134, light is emitted from the light diffusing element 108 to affect the formation of thrombi 120, either to hinder formation of the thrombi 120 or to promote the formation of thrombi 120 (in terms of speed of reaction and/or strength of bonds) to prevent them from breaking away from the intravenous catheter 102.

Advantageously, the method 130 and illumination system 100 can be used to decrease morbidity and mortality of existing medical devices that are commonly used to treat, prevent, or diagnose disease. Further, the light diffusing element 108 can provide targeted light therapy, whereas other methods of treatment, such as blood thinners, circulate throughout the entire body and can affect a patient's ability to recover from other wounds. Still further, the flexible and thin light diffusing element 108 does not interfere with existing devices.

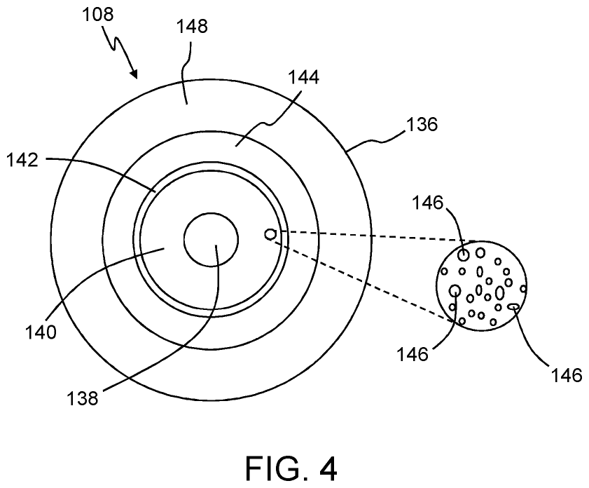

FIG. 4 depicts an embodiment of a light diffusing element 108 in the form of an LDF 136. In embodiments, the LDF 136 includes a glass or plastic core 138. The LDF 136 of FIG. 4 depicts a glass core 138 comprised of, e.g., pure or doped silica. A cladding layer 140 surrounds the core 138 along the longitudinal axis for all or a substantial portion of the length of the core 138. In embodiments, the LDF 136 also includes a coating layer 144 surrounding at least a portion of the cladding layer 140 along the longitudinal axis for at least a portion of the length of the cladding layer 140. The coating layer 144 may comprise a transparent medical grade polymer, a medical grade plastic, a plurality of photo-active sterilization molecules, a cross-linked coating, or a combination thereof. For example, the medical grade plastic may comprise silicone. The LDF 136 comprises a plurality of light scattering structures 146 distributed continuously or intermittently along at least a portion of the length of the LDF 136.

In embodiments, the light scattering structures 146 are positioned within the core 138, the cladding layer 140, or both. Further, the light scattering structures 146 are structurally configured such that the light diffusing optical fiber 136 emits light radially along the length of the LDF 136 when the therapeutic light source 106 emits light. For example, the LDF 136 may radially emit light at a scattering induced attenuation loss comprising between about 0.1 dB/m and about 100 dB/m, for example, at about 0.5 dB/m, 1 dB/m, 5 dB/m, 10 dB/m, 25 dB/m, 50 dB/m, 75 dB/m, or the like.

Further, the LDF 136 may comprise a diameter between about 100 μm and about 500 μm, for example, about 200 μm, about 250 μm, about 300 μm, or the like, and may comprise a bend radius between about 5 mm and about 15 mm. for example, 7 mm, 10 mm, 12 mm, or the like.

In embodiments, the light scattering structures 146 may comprise a plurality of gas filled voids or other nano-sized structures positioned within the core 138, the cladding layer 140, or both. The plurality of gas filled voids may be uniformly or non-uniformly distributed along the length of the LDF 136. In operation, the plurality of gas filled voids scatter a portion of the light traversing the length of the LDF 136 outward from the LDF 136. The plurality of gas filled voids may be positioned within LDF 136 such that a cross section of the LDF 136 may contain 50 or more gas filled voids, for example, 75 or more, 100 or more, or 200 or more. In operation, an increased number of gas filled voids positioned within the LDF 136 may generate an increased scattering induced attenuation loss when light traverses the LDF 136. Further, the plurality of gas filled voids may house any gas, for example, SO₂, Kr, Ar, CO₂, N₂, O₂ or a mixture thereof. Moreover, the cross-sectional size (e.g., diameter) of each of the plurality of gas filled voids may be between about 10 nm and about 1 μm, for example, between about 15 nm and about 500 nm, or the like.

The light scattering structures 146 may also comprise a refractive coating 142 optically coupled to a core 138 of the LDF 136. The refractive coating 142 may be positioned on the cladding layer 140, for example, surrounding or intermittently positioned on the cladding layer 140 or may be positioned directly on the core 138, for example, surrounding or intermittently positioned on the core 138. Further, the coating layer 144 may comprise the refractive coating, the refractive coating 142 may be positioned between the cladding layer 140 and the coating layer 144, the refractive coating may surround the coating layer 144, or the refractive coating may be intermittently positioned on the coating layer 144.

Further, the refractive coating 142 comprises an index of refraction that is greater than an index of refraction of the core 138 and the index of refraction of the cladding layer 140 such that at least partial refraction may occur at the optical interface formed between the refractive coating and the core 138, cladding layer 140, or the like, such that at least a portion of light traversing the core 138 exits the core 138, traverses the refractive coating, and scatters outward from the LDF 136. The refractive coating 142 may comprise any material having a higher index of refraction than the material of the core 138, such as GeO₂, TiO₂, ZrO₂, ZnO, BaS, alumina, or the like. For example, these higher index of refraction materials (e.g., GeO₂, TiO₂, ZrO₂, ZnO, BaS, alumina, or the like) may be particles (e.g., light scattering particles) dispersed within the refractive coating. Further, the light scattering particles may comprise a cross-sectional length, (e.g., diameter in embodiments comprising spherical particles) of between about 100 nm and about 2 μm, e.g., 250 nm, 500 nm, 750 nm, 1 μm, 1.5 μm, or the like. Moreover, the one or more light scattering structures 146 may comprise inks that include scattering pigments or molecules, such as TiO₂ positioned on or within the LDF 136. Other light scattering structures may include surface defect regions on the core 138, the cladding layer 140, or both.

As depicted in FIG. 4, the LDF 136 may also include a fiber jacket 148 surrounding the coating layer 144 (if provided). The fiber jacket 148 may comprise a transparent medical grade polymer, a cross-linked coating, or a combination thereof. A portion of the fiber jacket 148 may be opaque such that light does not emit radially along the opaque portion of the fiber jacket 148.

For the purposes of describing and defining the present invention it is noted that the term “about” is utilized herein to represent the inherent degree of uncertainty that may be attributed to any quantitative comparison, value, measurement, or other representation. The term “about” is also utilized herein to represent the degree by which a quantitative representation may vary from a stated reference without resulting in a change in the basic function of the subject matter at issue. In any of the various usages of “about” with respect to ranges of wavelength, dimensions of the LDF layers or scattering particles, etc., the uncertainty surrounding the end points of the range may be within 5%, more particularly within 2%, and even more particularly within 1%. Similarly, where used, the term “substantially” or “approximately” may provide a degree of deviation of, e.g., up to 5%, up to 2%, or up to 1%.

Aspect (1) of this disclosure pertains to a method, comprising the steps of: providing an intravenous catheter, wherein the intravenous catheter comprises an inner surface and an outer surface; inserting a light diffusing element into the intravenous catheter; emitting light from the light diffusing element such that the light irradiates the intravenous catheter; wherein the light emitted from the light diffusing element is configured to promote or hinder thrombi formation on the inner surface or outer surface of the intravenous catheter.

Aspect (2) of this disclosure pertains to the method of Aspect (1), wherein the light diffusing element comprises a light-diffusing optical fiber having a core surrounded by a cladding.

Aspect (3) of this disclosure pertains to the method of Aspect (2), wherein the core comprises at least one of a glass or a plastic.

Aspect (4) of this disclosure pertains to the method of Aspect (2) or Aspect (3), wherein the cladding comprises at least one of a glass or a plastic.

Aspect (5) of this disclosure pertains to the method of any one of Aspects (1) through (4), wherein the light diffusing element comprises a plurality of light-emitting diodes periodically spaced along a length of the light diffusing element.

Aspect (6) of this disclosure pertains to the method of Aspect (5), wherein the light-emitting diodes are substantially evenly spaced along the length of the light diffusing element.

Aspect (7) of this disclosure pertains to the method of any one of Aspects (1) through (6), wherein the light diffusing element irradiates the inner surface of the intravenous catheter.

Aspect (8) of this disclosure pertains to the method of Aspect (7), wherein the catheter is transparent to the light diffused from the light diffusing element and wherein the light diffusing element irradiates the outer surface of the intravenous catheter through the inner surface of the intravenous catheter.

Aspect (9) of this disclosure pertains to the method of any one of Aspects (1) through (8), wherein the light emitted from the light diffusing element is ultraviolet light having a wavelength of 200 nm to 400 nm.

Aspect (10) of this disclosure pertains to the method of any one of Aspects (1) through (8), wherein the light emitted from the light diffusing element is infrared light having a wavelength of 800 nm to 2000 nm.

Aspect (11) of this disclosure pertains to the method of any one of Aspects (1) through (8), wherein the light emitted from the light diffusing element has a wavelength of from 350 nm to 800 nm.

Aspect (12) of this disclosure pertains to the method of Aspect (11), wherein the intravenous catheter is located within a blood vessel, and wherein the method further comprises stimulating a photosensitizer in the blood vessel.

Aspect (13) of this disclosure pertains to the method of any one of Aspects (1) through (8), wherein the light emitted from the light diffusing element has a wavelength of from 400 nm to 1310 nm and an intensity of 5 to 500 mW/cm².

Aspect (14) of this disclosure pertains to the method of any one of Aspects (1) through (13), wherein the light emitted hinders thrombi formation and wherein the method further comprises preventing thrombi from forming on at least one of the inner surface or the outer surface of the catheter.

Aspect (15) of this disclosure pertains to the method of any one of Aspects (1) through (13), wherein the light emitted promotes thrombi formation and wherein the method further comprises preventing emboli from detaching from at least one of the inner surface or the outer surface of the catheter.

Aspect (16) of this disclosure pertains to the method of any one of Aspects (1) through (15), wherein the catheter comprises a photo-active coating on the inner surface or the outer surface and wherein the light emitted from the light diffusing element causes the photo-active coating to emit a secondary light having a wavelength in a wavelength range of 200 nm to 400 nm, 800 nm to 2000 nm, 350 nm to 800 nm, or 400 nm to 1310 nm.

Aspect (17) of this disclosure pertains to the method of any one of Aspects (1) through (15), wherein the catheter comprises a photo-active coating on the inner surface or the outer surface and wherein the light emitted from the light diffusing element causes the photo-active coating to become hydrophilic or hydrophobic.

Aspect (18) of this disclosure pertains to an illumination system, comprising: a light source configured to emit light having a wavelength of from 200 nm to 2000 nm; a light diffusing element optically coupled to the light source, wherein the light diffusing element is configured to receive the light emitted by the light source and diffuse the light along a length thereof and wherein the light diffusing element is configured to be inserted into, embedded in, or attached to an intravenous catheter located in a blood vessel; wherein the light diffused from the light diffusing element is configured to promote or hinder the formation of thrombi on an inner surface or an outer surface of the intravenous catheter.

Aspect (19) of this disclosure pertains to the illumination system of Aspect (18), wherein the light source comprises at least one of a light-emitting diode, a laser, an incandescent lamp, or a laser diode.

Aspect (20) of this disclosure pertains to the illumination system of Aspect (18) or Aspect (19), further comprising the intravenous catheter, wherein the light diffusing element is configured to be reversibly inserted into a central bore defined by the inner surface of the intravenous catheter.

Aspect (21) of this disclosure pertains to the illumination system of Aspect (18) or Aspect (19), further comprising the intravenous catheter, wherein the light diffusing element is attached to the inner surface or to the outer surface of the intravenous catheter.

Aspect (22) of this disclosure pertains to the illumination system of Aspect (18) or Aspect (19), further comprising the intravenous catheter, wherein the light diffusing element is embedded between the inner surface and the outer surface of the intravenous catheter.

Aspect (23) of this disclosure pertains to the illumination system of any one of Aspects (20) through (22), wherein the intravenous catheter further comprises a photo-active coating disposed on at least one of the inner surface or the outer surface of the intravenous catheter.

Aspect (24) of this disclosure pertains to the illumination system of Aspect (23), wherein, upon being illuminated by the light diffused from the light diffusing element, the photo-active coating emits a secondary light having a wavelength different from the light diffused by the light diffusing element.

Aspect (25) of this disclosure pertains to the illumination system of Aspect (23), wherein, upon being illuminated by the light diffused from the light diffusing element, the photo-active coating becomes hydrophilic or hydrophobic.

Aspect (26) of this disclosure pertains to the illumination system of any one of Aspect (18) through (25), wherein the light diffusing element comprises a light-diffusing optical fiber having a core surrounded by a cladding.

Aspect (27) of this disclosure pertains to the illumination system of Aspect (26), wherein the core comprises at least one of a glass or a plastic.

Aspect (28) of this disclosure pertains to the illumination system of Aspect (26) or Aspect (27), wherein the cladding comprises at least one of a glass or a plastic.

Aspect (29) of this disclosure pertains to the illumination system of any one of Aspects (18) through (28), wherein the light diffused from the light diffusing element is ultraviolet light having a wavelength of 200 nm to 400 nm.

Aspect (30) of this disclosure pertains to the illumination system of any one of Aspects (18) through (28), wherein the light diffused from the light diffusing element is infrared light having a wavelength of 800 nm to 2000 nm.

Aspect (31) of this disclosure pertains to the illumination system of any one of Aspects (18) through (28), wherein the light diffused from the light diffusing element has a wavelength of from 350 nm to 800 nm and is configured to activate a photosensitizer in the blood vessel.

Aspect (32) of this disclosure pertains to the illumination system of any one of Aspects (18) through (28), wherein the light diffused from the light diffusing element has a wavelength of from 400 nm to 1310 nm and an intensity of 5 to 500 mW/cm².

Aspect (33) of this disclosure pertains to an indwelling catheter system, comprising: a catheter configured for insertion into a blood vessel, the catheter having an inner surface and an outer surface, the inner surface defining a central bore extending along a longitudinal axis of the catheter; a light diffusing element disposed within the central bore of the catheter; and a light source optically coupled to the light diffusing element and configured to emit light; wherein the light diffusing element is configured to receive the light emitted by the light source and diffuse the light along a length thereof; and wherein the light diffused from the light diffusing element is configured to promote or hinder thrombi formation on the inner surface or the outer surface of the catheter.

Aspect (34) of this disclosure pertains to the indwelling catheter system of Aspect (33), wherein the light source comprises at least one of a light-emitting diode, a laser, or a laser diode.

Aspect (35) of this disclosure pertains to the indwelling catheter system of Aspect (33) or Aspect (34), wherein the light diffusing element is configured to be reversibly inserted into the central bore of the catheter.

Aspect (36) of this disclosure pertains to the indwelling catheter system of any one of Aspects (33) through (35), wherein the catheter further comprises a photo-active coating disposed on at least one of the inner surface or the outer surface.

Aspect (37) of this disclosure pertains to the indwelling catheter system of Aspect (36), wherein, upon being illuminated by the light diffused from the light diffusing element, the photo-active coating emits a secondary light having a wavelength different from the light diffused by the light diffusing element.

Aspect (38) of this disclosure pertains to the indwelling catheter system of Aspect (36), wherein, upon being illuminated by the light diffused from the light diffusing element, the photo-active coating becomes hydrophilic or hydrophobic.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred. In addition, as used herein, the article “a” is intended to include one or more than one component or element, and is not intended to be construed as meaning only one.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A method, comprising the steps of:
providing an intravenous catheter, wherein the intravenous catheter comprises an inner surface and an outer surface;
inserting a light diffusing element into the intravenous catheter;
emitting light from the light diffusing element such that the light irradiates the intravenous catheter;
wherein the light emitted from the light diffusing element is configured to promote or hinder thrombi formation on the inner surface or outer surface of the intravenous catheter.

2. The method of claim 1, wherein the light diffusing element comprises a light-diffusing optical fiber having a core surrounded by a cladding.

3. The method of claim 2, wherein the core comprises at least one of a glass or a plastic.

4. The method of claim 2, wherein the cladding comprises at least one of a glass or a plastic.

5. The method of claim 1, wherein the light diffusing element comprises a plurality of light-emitting diodes periodically spaced along a length of the light diffusing element.

6. The method of claim 5, wherein the light-emitting diodes are substantially evenly spaced along the length of the light diffusing element.

7. The method of claim 1, wherein the light diffusing element irradiates the inner surface of the intravenous catheter.

8. The method of claim 7, wherein the catheter is transparent to the light diffused from the light diffusing element and wherein the light diffusing element irradiates the outer surface of the intravenous catheter through the inner surface of the intravenous catheter.

9. The method of claim 1, wherein the light emitted from the light diffusing element is ultraviolet light having a wavelength of 200 nm to 400 nm.

10. The method of claim 1, wherein the light emitted from the light diffusing element is infrared light having a wavelength of 800 nm to 2000 nm.

11. The method of claim 1, wherein the light emitted from the light diffusing element has a wavelength of from 350 nm to 800 nm.

12. The method of claim 11, wherein the intravenous catheter is located within a blood vessel, and wherein the method further comprises stimulating a photosensitizer in the blood vessel.

13. The method of claim 1, wherein the light emitted from the light diffusing element has a wavelength of from 400 nm to 1310 nm and an intensity of 5 to 500 mW/cm².

14. The method of claim 1, wherein the light emitted hinders thrombi formation and wherein the method further comprises preventing thrombi from forming on at least one of the inner surface or the outer surface of the catheter.

15. The method of claim 1, wherein the light emitted promotes thrombi formation and wherein the method further comprises preventing emboli from detaching from at least one of the inner surface or the outer surface of the catheter.

16. The method claim 1, wherein the catheter comprises a photo-active coating on the inner surface or the outer surface and wherein the light emitted from the light diffusing element causes the photo-active coating to emit a secondary light having a wavelength in a wavelength range of 200 nm to 400 nm, 800 nm to 2000 nm, 350 nm to 800 nm, or 400 nm to 1310 nm.

17. The method of claim 1, wherein the catheter comprises a photo-active coating on the inner surface or the outer surface and wherein the light emitted from the light diffusing element causes the photo-active coating to become hydrophilic or hydrophobic.

18. An illumination system, comprising:
a light source configured to emit light having a wavelength of from 200 nm to 2000 nm;
a light diffusing element optically coupled to the light source, wherein the light diffusing element is configured to receive the light emitted by the light source and diffuse the light along a length thereof and wherein the light diffusing element is configured to be inserted into, embedded in, or attached to an intravenous catheter located in a blood vessel;
wherein the light diffused from the light diffusing element is configured to promote or hinder the formation of thrombi on an inner surface or an outer surface of the intravenous catheter.

19. The illumination system of claim 18, wherein the light source comprises at least one of a light-emitting diode, a laser, an incandescent lamp, or a laser diode.

20. The illumination system of claim 18, further comprising the intravenous catheter, wherein the light diffusing element is configured to be reversibly inserted into a central bore defined by the inner surface of the intravenous catheter.

21. The illumination system of claim 18, further comprising the intravenous catheter, wherein the light diffusing element is attached to the inner surface or to the outer surface of the intravenous catheter.

22. The illumination system of claim 18, further comprising the intravenous catheter, wherein the light diffusing element is embedded between the inner surface and the outer surface of the intravenous catheter.

23. The illumination system of claim 20, wherein the intravenous catheter further comprises a photo-active coating disposed on at least one of the inner surface or the outer surface of the intravenous catheter.

24. The illumination system of claim 23, wherein, upon being illuminated by the light diffused from the light diffusing element, the photo-active coating emits a secondary light having a wavelength different from the light diffused by the light diffusing element.

25. The illumination system of claim 23, wherein, upon being illuminated by the light diffused from the light diffusing element, the photo-active coating becomes hydrophilic or hydrophobic.

26. The illumination system of claim 18, wherein the light diffusing element comprises a light-diffusing optical fiber having a core surrounded by a cladding.

27. The illumination system of claim 26, wherein the core comprises at least one of a glass or a plastic.

28. The illumination system of claim 26, wherein the cladding comprises at least one of a glass or a plastic.

29. The illumination system of claim 18, wherein the light diffused from the light diffusing element is ultraviolet light having a wavelength of 200 nm to 400 nm.

30. The illumination system of claim 18, wherein the light diffused from the light diffusing element is infrared light having a wavelength of 800 nm to 2000 nm.

31. The illumination system of claim 18, wherein the light diffused from the light diffusing element has a wavelength of from 350 nm to 800 nm and is configured to activate a photosensitizer in the blood vessel.

32. The illumination system of claim 18, wherein the light diffused from the light diffusing element has a wavelength of from 400 nm to 1310 nm and an intensity of 5 to 500 mW/cm².

33. An indwelling catheter system, comprising:
a catheter configured for insertion into a blood vessel, the catheter having an inner surface and an outer surface, the inner surface defining a central bore extending along a longitudinal axis of the catheter;
a light diffusing element disposed within the central bore of the catheter; and
a light source optically coupled to the light diffusing element and configured to emit light;
wherein the light diffusing element is configured to receive the light emitted by the light source and diffuse the light along a length thereof; and
wherein the light diffused from the light diffusing element is configured to promote or hinder thrombi formation on the inner surface or the outer surface of the catheter.

34. The indwelling catheter system of claim 33, wherein the light source comprises at least one of a light-emitting diode, a laser, or a laser diode.

35. The indwelling catheter of claim 33, wherein the light diffusing element is configured to be reversibly inserted into the central bore of the catheter.

36. The indwelling catheter system of claim 33, wherein the catheter further comprises a photo-active coating disposed on at least one of the inner surface or the outer surface.

37. The indwelling catheter system of claim 36, wherein, upon being illuminated by the light diffused from the light diffusing element, the photo-active coating emits a secondary light having a wavelength different from the light diffused by the light diffusing element.

38. The indwelling catheter system of claim 36, wherein, upon being illuminated by the light diffused from the light diffusing element, the photo-active coating becomes hydrophilic or hydrophobic.
